(19) United States
(12) Patent Application Publication
(10) Pub. No.: US 2004/0016028 A1
(43) Pub. Date: Jan. 22, 2004

(54) Method for generating or increasing resistance to biotic or abiotic stress factors in organisms

(76) Inventors: Ekkehard Neuhaus, Im Braumenstuck (DE); Klaus During, Vorgebirgsweg (DE)

Correspondence Address: HELLER EHRMAN WHITE & MCAULIFFE LLP, 1666 K STREET,NW, SUITE 300, WASHINGTON, DC, 20006, US

(21) Appl. No.: 10/381,732

(22) PCT Filed: Sep. 26, 2001
(86) PCT No.: PCT/DE01/03768

(30) Foreign Application Priority Data
Sep. 28, 2000 (DE) 100 49 267.3

(51) Int. Cl. A01H 1/00; C12N 15/82

(52) U.S. Cl. 800/289; 800/279

(57) ABSTRACT

The invention relates to a method of generating or increasing a resistance in organisms, in particular plants, to biotic and abiotic stress. The method is based on a change adapted to be carried out by various methods in the distribution of ATP and/or ADP in cells of the organism.

The present invention relates to a method of generating or increasing a resistance to biotic and abiotic stress in organisms, in particular plants. The method is based on a change, adapted to be carried out using various methods, in the distribution of ATP and/or ADP within the cells of the organisms.

Plants are exposed to a number of biotic and abiotic stress factors. The biotic stress factors comprise above all pathogens, e.g. phytopathogenic fungi, bacteria and viruses, while the abiotic stress factors comprise in particular heat, cold, dryness and salt stress. The yield of the agricultural or hortocultural cultivation of the cultivated plants is affected considerably by these stress factors or even whole harvests are destroyed. For a long time, classical plant breeding has therefore tried to integrate resistance to biotic and abiotic stress factors, in particular to pathogens, into the current plant varieties. Known effective resistances, in particular in the case of disease resistances, are usually resistance mechanisms based on the interplay of a number of involved genes which are often also distributed over several chromosomes so that the development of efficiently resistant varieties is very difficult. In addition, in many cases there are no naturally occurring resistance mechanisms in the available gene pool. Other resistance features are again ineffective so that no adequate or lasting protection can be reached.

It has thus been tried for many years to fill the gaps in plant breeding by using chemical crop protection agents. However, this requires the large scale use of chemicals usually harmful to the environment in the field. In many cases, the use of genetic engineering by means of which it is tried to introduce new resistance genes or improve known resistance mechanisms, has not yet yielded the expected success.

The present invention is thus based on the technical problem of providing a product by which wide, general resistance to biotic and abiotic stress can be generated in organisms, in particular plants.

This technical problem is solved by the subject matters defined in the claims. The present invention comprises a new resistance mechanism to biotic and abiotic stress factors in organisms, such as plants, which is based on an increase in the general resistance. It has been found surprisingly that by modifying the distribution of ATP or ADP within the cell it is possible to induce a physiological change so as to achieve a significantly higher resistance, e.g. to plant pests.

ATP is the universal energy carrier of all living cells. Energy in the form of ATP is required for almost all anabolic metabolic pathways. In heterotrophic plant cells, ATP is mainly synthesized from ADP and inorganic phosphate within the mitochondria by means of oxidative phosphorylation. Under anaerobic conditions, this is effected by means of substrate-level phosphorylation in the cytosol. ATP is transported out of the mitochondria by means of the mitochondrial ADP/ATP transport protein which is one of the best-studied membrane proteins. The mitochondrial ADP/ATP transport protein catalyzes exclusively the export of ATP in return for the import of ADP.

In the case of heterotrophic vegetable storage tissues a comparatively large amount of ATP is taken up into the storage plastids to energize biosynthesis steps which only occur there, as for the starch or fatty acid biosynthesis. This uptake is catalyzed by a plastidial ATP/ADP transport protein localized within the inner coat membrane and enabling the ATP uptake in return for the ADP release.

In order to analyze the effect of modified plastidial ATP/ADP transporter activities on the carbohydrate balance, transgenic potato plants having an increased or reduced transport activity were produced by the experiments resulting in the present invention.

The amount of the endogenous plastidial ATP/ADP transporter in potatoes (AATP1, Solanum tuberosum St) was reduced by means of antisense inhibition. Part of the cDNA coding for AATP1,St was introduced into the potato genome in antisense orientation, different independent lines each having individually reduced activity of the plastidial ATP/ADP transporter having been obtained. This cDNA was controlled by the constitutive cauliflower mosaic virus 35S promoter. The activity of the plastidial ATP/ADP transporter was thus reduced to 64% to 79% as compared to that of non-transgenic control plants. The transgenic potato plants showed no phenotypic changes in the aboveground green tissues. On the contrary, the morphology of the tubers was markedly altered (branched tubers) and the starch content dropped to about 50% as compared to the non-transgenic control plants (Tjaden et al., Plant Journal, 16 (1998), 531-540). Correspondingly, this physiological finding means that on account of the reduced ATP/ADP transporter activity the plastids took up a comparatively reduced amount of ATP.

Transgenic potato plants having an increased activity of the plastidial ATP/ADP transporter were also produced by introducing the cDNA for the plastidial ATP/ADP transporter from Arabidopsis thaliana (AATP1,At) into the potato genome in a sense orientation under the control of the 35S promoter. As a result, various independent lines formed each showing an individually increased activity of the plastidial ATP/ADP transporter. In the different lines, the measured activity of the plastidial ATP/ADP transporter was between 130 and 148% as compared to that in non-transgenic control plants. The transgenic potato plants showed no phenotypic changes in the aboveground green tissues. However, the starch content was increased by up to about 150% of the non-transgenic control tubers (Tjaden et al., supra). This physiological finding thus means that on account of the increased ATP/ADP transporter activity the plastics took up comparatively increased amount of ATP.

It has to be assumed that the change in the ATP or ADP concentrations in certain plant cell portions has considerable effects on the cell metabolism and the regulation of genes. The studies resulting in the present invention thus served for investigating whether the resistance properties of the plants are also influenced by such a change. To this end, transgenic potato plants of the Desirée variety were produced e.g. by means of the gene constructs described in Tjaden et al. (supra) to lower the antisense or raise the sense of the ATP/ADP transporter activity. They were checked phytopathologically as to their resistance properties. For this purpose, in particular the resistance to the phytopathogenic bacterium Erwinia carotovora was tested intensively in tuber slide tests. It turned out that the resistance properties of the transgenic plants were markedly improved (cf. below Example 1 and FIG. 1).

The present invention thus relates to a method of creating or increasing a resistance of organisms, preferably plants, to biotic or abiotic stress factors, which is characterized by changing the distribution of ATP and/or ADP in cells of the organisms (as compared to the original situation).

The term “resistance to biotic or abiotic stress factors” as used herein relates to a resistance to a number of factors referred to as biotic or abiotic stress factors. The biotic stress factors to be mentioned are in particular phytopathogenic fungi, such as Phytophthora infestans, Botrytis cinerea, Alternaria alternata, Fusarium oxysporum, Ustilago maydis, and bacterial pathogens, such as Erwinia carotovora, Pseudomonas syringae, Ralstonia solanacearum, Xanthomonas campestris and Clavibacter michiganense. Abiotic stress factors to be mentioned are in particular cold, heat, dryness, U.V. radiation and salt stress. The resistance obtained by the method according to the invention is thus preferably a disease resistance, pest resistance.

The organisms suitable for the method according to the invention are animals, humans and plants. Plants may be, in principle, plants of any plant variety, i.e. both monokotyl and dikotyl plants contain one or more transgenes and express them parallel or sequentially. The parallel expression of several transgenes is conceivable via the control of the coding sequences by constitutive and/or inducible promoters. A sequential expression can be achieved by the regulation of the gene expression of several transgenes in an organism, which can be induced in different ways.

The organisms suitable for the method according to the invention are animals, humans and plants. The plants may, in principle, be plants of any plant species, i.e. both monocotyl and dicotyl plants. The term “plant” as used herein also comprises gramineae, chenopodiums, leguminous plants, brassicaceae, solanaceae, fungi, mosses, and algae. Useful plants, e.g. plants such a wheat, barley, rice, corn, sugar beets, sugarcane, rape, mustard, oilseed rape, flax, peas, beans, lupins, tobacco and potatoes are particularly preferred.

In a preferred embodiment, the method according to the invention is characterized by increasing or reducing in the organism the activity or concentration of a protein which is involved in the subcellular distribution of ATP and ADP, a protein being concerned which is available in the corresponding organism by nature, e.g. the plastidial ATP/ADP transporter or the plastidial triose phosphate/phosphate transporter. An embodiment of the method according to the invention is particularly preferred in which the expression of a gene coding for such a protein is increased or reduced. This gene expression can be modified by means of methods known to a person skilled in the art. For example, this can be effected by the protein concentration change described above and in Example 1 using antisense or sense constructs. Basically, the protein activity or concentration can be changed both on the gene expression level and via a functional inhibition of the protein activity, e.g. by the expression of binding, inhibiting, neutralizing or catalytic antibodies or other specifically binding and blocking proteins or peptides, by ribozymes, single-stranded or double-stranded oligonucleotides, aptamers, lipids, natural receptors, lectins, carbohydrates, etc.

In the method according to the invention, the ATP or ADP concentration in cell compartments can also be influenced by introducing a protein (polypeptide) which is not available in the respective organism by nature. In order to obtain the localization of the protein in the desired cell compartment it may be favorable for the protein to have a signal peptide, so that it can be transported into certain cell compartments of a plant cell. The person skilled in the art is familiar with suitable signal peptides and methods of linking the signal peptides with a desired protein. For example, reference is made to the signal peptide of amylase from barley as regards the apoplast (Düring et al., Plant Journal 3 (1993), 587-598), to a murine signal peptide, to the combination between a murine signal peptide and the KDEL-ER retention signal as to ER (Artsaenko et al., Molecular Breeding 4 (1998), 313-319), to the targeting signal of a mammal-2,6-sialyltransferase regarding the Golgi apparatus (Wee et al., Plant Cell IV (1998), 1759-1768), to the vacuol localizing signal of a vacuolar chitinase from cucumber as regards the vacuols (Neuhaus et al., Proc. Natl. Acad. Sci. U.S.A. 88 (1991), 10362-10366), to the ferredoxin transit peptide regarding the chloroplasts and plastids, and to the transit peptide of tryptophanyl tRNA synthethase from yeast as to the mitochondria (Schmitz and Lonsdale, Plant Cell 1 (1998), 783-791). Basically, the protein involved in the subcellular distribution of ATP and ADP can be administered by various methods, e.g. via media, such as the culture media, of a plant or of parts thereof, in particular plant cells. However, as pointed out above already, it is preferred to give the plants or portions thereof the protein in the form of a nucleic acid coding for it, e.g. DNA or RNA. For this purpose, it is necessary for the nucleic acid to be available in an expression vector or to be ligated with sequences thereof. In this connection, it may be favorable for this vector or these sequences to enable an expression of the nucleic acid in cell compartments. Such expression vectors or sequences thereof are known to the person skilled in the art. For example, reference is made to Svab et al., Proc. Natl. Acad. Sci. U.S.A. 87 (1990), 8526-8530; Khan and Maliga, Nature Biotechnology 17 (1999), 910-915; and Sidorov et al., Plant Journal 19 (1999), 209-216.

Methods of constructing the expression vectors containing the desired gene, e.g. for a plastidial ATP/ADP transporter from Arabidopsis thaliana (AATP1,At) in an expressible form are known to the person skilled in the art and also described in common standard works, for example (cf. e.g. Sambrook et al., 1989, Molecular Cloning, A Laboratory Manual, 2^{nd} edition, Cold Spring Harbor Laboratory Press, Cold Spring Harbor, N.Y.). The expression vectors can be based on a plasmid, cosmid, virus, bacteriophage or another vector common in genetic engineering. These vectors may have further functional units which effect stabilization of the vector in the plants, for example. AS regards plants they may contain left-border and right-border sequences of agrobacterial T-DNA so as to enable stable integration into the genotype of plants. A termination sequence may also be present which serves for correctly terminating the transcription and the addition of a poly-A sequence to the transcript. Such elements are described in the literature (cf. Gielen et al., EMBO J. 8 (1989), 23-29) and can be exchanged as desired.

The person skilled in the art is familiar with promoters suited for the expression of the gene coding for the desired protein. These promoters include e.g. the cauliflower mosaic virus 35S promoter (Odell et al., Nature 313 (1995), 810-812), the Agrobacterium tumefaciens nopaline synthase promoter and the mannopine synthase promoter (Harpster et al., Molecular and General Genetics 212 (1988), 182-190).

The increase or decrease of the above-described protein activities can be effected constitutively or temporally, locally or be induced by certain stimuli. A temporally or locally limited or inducible increase or decrease in the protein activities also suppresses the changes in the tuber morphology, described by Tjaden et al. (supra).

Thus, another preferred embodiment of the method according to the invention is characterized by regulating the expression of the desired gene temporally, locally or inducibly in the organism. For example, the gene coding for the desired protein can be linked to an inducible promoter, which permits e.g. the control of the synthesis of the desired protein, e.g. in a plant, at a desired time. Suitable promoters are known to the person skilled in the art and comprise e.g. the anaerobically inducible Gap C4 promoter from corn (Bülow et al., Molecular Plant-Microbe Interactions 12 (1999), 182-188), PR promoters such as L-phenylalanine ammonium lyase, chalcon synthase and hydroxyproline rich glycoprotein promoters, inducible by ethylene (Ecker and Davies, Proc. Natl. Acad. Sci. U.S.A. 84 (1987), 5202-5210) and a dexamethasone-inducible chimeric transcription induction system (Kunkel et al., Nature Biotechnology 17 (1990), 916-918), the IncW promoter from corn inducible by saccharose or D-glucose (Chen et al., Proc. Natl. Acad. Sci. U.S.A. 96 (1999), 10512-10517). Reference is also made to Dalta et al., Biotechnology Annual Review 3 (1997), 269-290, and Gatz and Denk, Trends in Plant Science 3 (1998), 352-358. Furthermore, suitable promoters permit a local regulation of the expression, i.e. only in certain plant parts or organs. Such promoters are e.g. the patatin promoter from potatoes (Liu et al., Molecular and General Genetics 223 (1990), 401-406) (tuber-specific), the napin promoter from rape (Radke et al., Theoretical and Applied Genetics 75 (1988), 685-694) (embryo-specific in the seed), the RolC promoter from Agrobacterium rhizogenes (Yokoyama et al., Molecular and General Genetics 244 (1994), 15-22) (phloem-specific), the TA29 promoter from tobacco (Kriete et al., Plant Journal 9 (1996), 809-818) (tapetum-specific), the LeB4 promoter from Vicia faba (Bäumlein et al., Molecular and General Genetics 225 (1991), 121-128) (seed-specific) and the rbcS and cab promoters from petunia (Jones et al., Molecular and General Genetics 212 (1988), 536-542) (leaf-specific or limited to photosynthetically active tissues).

In another preferred embodiment of the method according to the invention, the expression of the plastidial ATP/ADP transporter is raised or lowered. In this connection, the expression can be lowered by introducing an antisense construct suppressing the expression of the endogenous gene, and the expression can be raised by introducing a sense construct. The sense construct may be a gene available on an expression vector for the endogenous transporter e.g. under the control of a strong promoter but also a heterologous gene coding for a transporter from another organism, preferably a closely related organism.

A large number of cloning vectors which contain a replication signal for E. coli and a marker gene for the selection of transformed bacterial cells are available for the production of the expression vectors which shall be introduced into plants. Examples of such vectors are pBR322, pUC series, M13mp series, pA-CYC184, etc. The desired sequence may be introduced into the vector at an appropriate restriction site. The resulting vector is used for the transformation of E. coli cells. Transformed E. coli cells are cultured in a suitable medium, then harvested and lyzed. The vector is then recovered. In general, restriction analyses, gel electrophoreses and further biochemically molecular-biological methods are used as analytical methods for characterizing the vector DNA obtained. Following every manipulation, the vector DNA can be cleaved and the DNA fragments obtained can be linked with other DNA sequences. Each vector DNA sequence can be cloned into the same or into other vectors.

A number of methods are available for the introduction of the above expression vectors into a plant cell. These methods comprise transformation of plant cells with T-DNA using Agrobacterium tumefaciens or Agrobacterium rhizogenes as transformation means, the fusion of protoplasts, the injection, the electroporation of DNA, the introduction of DNA by means of the biolistic method and further possibilities.

The injection and electroporation of DNA in plant cells do generally not make special demands on the employed vectors. It is possible to use simple plasmids such as pUC derivatives. However, if whole plants shall be regenerated from cells transformed in this way, a selectable marker should be present. Suitable selectable markers are known to the person skilled in the art and comprise e.g. the neomycin phosphotransferase II gene from E. coli (Beck et al., Gene 19 (1982), 327-336), the sulfonamide resistance gene (EP-369637), and the hygromycin resistance gene (EP-186425). Depending on the method of introducing the desired gene into the plant cell, further DNA sequences may be required. For example, if the Ti or Ri plasmid is used for the transformation of the plant cell, at least the right boundary, but often the right and left boundaries, of the Ti and Ri plasmid T-DNA have to be connected as a flange region with the genes to be introduced.

If agrobacteria are used for the transformation, the DNA to be introduced must be cloned into special vectors, i.e. into either an intermediary vector or a binary vector (cf. below Example 1). Due to sequences homologous to sequences in the T-DNA, the intermediary vectors can be integrated into the Ti or Ri plasmid of the agrobacteria by homologous recombination. It also contains the vir region necessary for the T-DNA transfer. Intermediary vectors cannot replicate in agrobacteria. By means of a helper plasmid, the intermediary vector can be transferred to Agrobacterium tumefaciens. Binary vectors can replicate in both E. coli and agrobacteria. They contain a selection marker gene and a linker or polylinker, which are surrounded by the right and left T-DNA boundary regions. They can be transformed directly into the agrobacteria. The agrobacterium serving as a host cell should contain a plasmid which carries a vir region. The vir region is necessary for the transfer of T-DNA into the plant cell. Additional T-DNA may be present. The agrobacterium transformed in this way is used for the transformation of plant cells.

In order to transfer the DNA into the plant cell, plant explants can usefully be cocultured with Agrobacterium tumefaciens or Agrobacterium rhizogenes. Whole plants can then be regenerated again from the infected plant material (e.g. leaf portions, stem segments, roots, but also protoplasts or suspension-cultivated plant cells) in a suitable medium which may contain antibiotics of biocides for the selection of transformed cells. The resulting plants can subsequently be studied for the presence of the introduced DNA. Alternative systems for the transformation of monocotyl plants are the transformation by means of a biolistic approach, the electrically or chemically induced DNA uptake into protoplasts, the electroporation of partially permeabilized cells, the macroinjection of DNA into inflorescence, the microinjection of DNA into microspores and pro-embryos, the DNA uptake by germinating pollens, and the DNA uptake into embryos by swelling (for an overview see Potrykus, Biotechnologie 8 (1990), 535-542). While the transformation of dicotyl plants is well established via Ti plasmid vector systems using Agrobacterium tumefaciens, more recent studies indicate that monocotyl plants are also absolutely accessible to transformation by means of vectors based on agrobacterium.

In a preferred embodiment, the expression vectors used according to the invention contain localization signals for localizing them in cell compartments, in particular the endoplasmic reticulum (ER), apoplasts, Golgi apparatus, plastids, peroxisomes, mitochondria and/or vacuols. Reference is made to the above statements on the signal peptides. The KDEL-ER targeting peptide, the Golgi localization signal of β-1,2-N-acetylglucosamine transferase (GnTl), the transit peptide from the small subunit of ribulose bisphosphate carboxylase and/or the vacuolary targeting signal SKNPIN are particularly preferred as localization signals.

In principle, the plant portions desired for the expression of the protein relate to any plant portion, in any case to replication material of these plants, e.g. seeds, tubers or bulbs, rootstocks, seedlings, cuttings, etc.

In principle, by means of the present invention it is also possible to generate or increase a resistance to biotic and abiotic stress in animals and humans. For this purpose, the above protein can be administered as such or in combination with a signal peptide to animals, humans or cells thereof. Such a signal peptide may be e.g. a murine signal peptide, a combination of a murine signal peptide and the KDEL-ER retention signal, or the targeting signal of a mammal-alpha-2,6-sialyltransferase as regards the Golgi apparatus. Furthermore, the protein can be administered in the form of a nucleic acid coding for it, e.g. DNA or RNA, to animals, humans or cells thereof. Administration in the form of a nucleic acid requires that the latter is present in an expression vector or is ligated with sequences thereof. Reference is made to the above general statements on expression vectors and their production. By way of supplement, reference is made to vectors which are suited for the gene therapy in animals. In them, the nucleic acid can be controlled by an inducible or tissue-specific promoter, such as metallothionein I or polyhedrin promoter. Preferred vectors are e.g. viruses, such as retroviruses, adenoviruses, adeno-associated viruses or vaccinia viruses. Examples of retroviruses are MoMuLV, HaMuSV, MUMTV, RSV or GaLV. Furthermore, the nucleic acid coding for the polypeptide can be transported to the target cells in the form of colloidal dispersions. They comprise e.g. liposomes and lipoplexes (Mannino et al., Biotechniques 6 (1988), 682).

According to the invention, the above protein is administered to animals, humans and cells thereof. In principle, the animals may belong to any animal species. They are preferably useful and domestic animals, e.g. cattle, horses, sheep, pigs, goats, dogs, cats, etc.

Examples of biotic stress in animals or humans are in particular fungi pathogenic for animals, which produce diseases such as Candida infections, cryptococcoses, aspergilloses, dermatomycoses, hystoplasmoses, coccidiomycoses and blastomycoses, and bacterial pathogens such as micrococcaceae (e.g. staphylococci), lactobacteriaceae (e.g. streptococci), neisseriaceae (e.g. Neisseriae), corynebacteriaceae, spirillaceae, listeria bacteriae, mycobacteriaceae, enterobacteriaceae (e.g Escherichia bacteriae), salmonellae, brucellaceae (e.g. Pasteurella bacteriae), anaerobic and aerobic sporeforming bacteria (e.b. bacillaceae, clostridia), rickettsia. All in all, the methods according to the invention is suited in the best way to be used for the cultivation of plants and breeding of animals and in human medicine.

### BRIEF DESCRIPTION OF THE FIGURES

FIG. 1 shows remaining intact potato tuber tissue (in %) after the inoculation of tuber slices with 2000 Erwinia carotovora ssp. atroseptica bacteria in 2 μl and incubation for three days according to Düring et al., supra. Lines MPB/aATPT contain the antisense gene construct, lines MPB/sATPT contain the sense gene construct for the plastidial ATP/ADP transporter from Arabidopsis thaliana in transgenic potato plants of the Désirée variety. Désirée: non-transgenic starting variety as a control.

FIG. 2 shows the relative attack of leave tissue (in %) after the inoculation of leave slides with 20 μl spore suspension of Phytophthora infestans and incubation for five and six days. The lines MPB/aATP contain the antisense gene construct, lines MPB/sATP contain the sense gene construct for the plastidial ATP/ADP transporter from Arabidopsis thaliana in transgenic potato plants of the Désirée variety: non-transgenic starting variety as a control.

FIG. 3 is a picture showing the attack of potato plants infected with Phytophthora infestans after an incubation of 48 and 96 hours. The non-transgenic potato variety Désirée is referred to as WT. The designation AS was used for potato plants which carry the antisense gene construct for the plastidial ATP/ADP transporter form Arabidopsis thaliana.

The invention is explained by the below examples.

### EXAMPLE 1

### Increase in the Resistance of Transgenic Potato Tubers to Erwinia carotovora

The gene constructs described in Tjaden et al. (supra) for lowering the antisense (“MPB/aATPT”) or increasing the sense (“MBP/sATPT”) of the plastidial ATP/ADP transporter activity in potato tubers were ligated in each case in blunt-end fashion into the opened and filled singular HindIII restriction site of the binary vector pSR 8-30 (cf. Düring et al., supra; Porsch et al., Plant Molecular Biology (1998) 37, 581-585). The two transformation vectors pSR8-30/sATPT and pSR 8-30/sATPT were obtained. These two expression vectors were used separately for the transformation of E. coli SM10. Transformants were mixed with agrobacterium GV 3101 and incubated at 28° C. overnight. (Koncz and Schell, Mol. Gen. Genet. (1986) 204; 383-396, Koncz et al., Proc. Natl. Acad. Sci. U.S.A. (1987) 84, 131-135). Selection was made for carbenicillin, the bla gene necessary for this purpose being available in the above expression vectors. Selection clones of Agrobacterium tumefaciens were applied onto cut-off leaves, scratched several times at the middle rib, of potato plants cv. Désirée and the leaves were incubated at 20° C. in the dark for 2 days. Thereafter, the agrobacteria were washed off and plant growth substances were added to the potato leaves, so that preferably shoots regenerated. Furthermore, non-transformed cells were killed in the potato leaves by the addition of kanamycin to the plant medium. Growing shoots were cut off and were allowed to grow roots in the medium without plant growth substances but with kanamycin. The potato plants were further cultivated as usual. On the one hand, transgenic lines including the antisense gene construct and, on the other hand, transgenic lines including the sense gene construct were obtained. The regenerated potato lines were planted in mold and grown in a greenhouse. After the ripening of the potato plants, the tubers were harvested and stored for phytopathological examination.

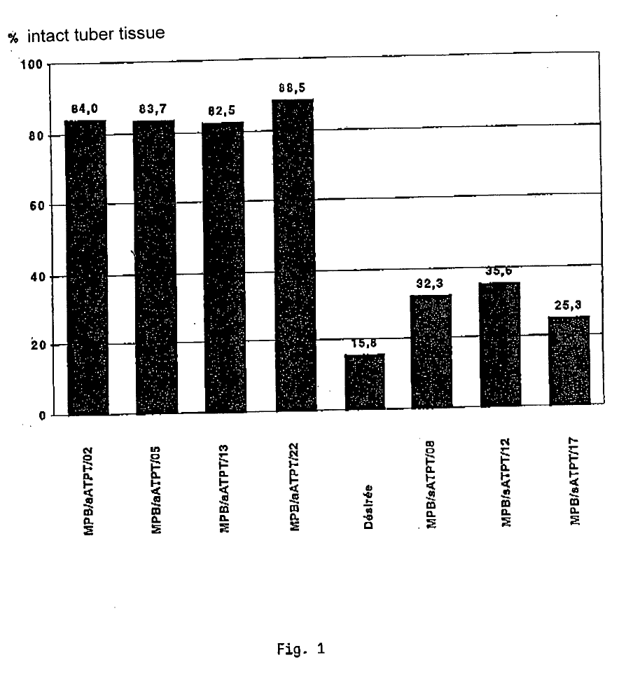

The resistance properties of the transgenic potato tubers to the bacterial pathogen Erwinia carotovora were checked in a tuber slice experiment. For this purpose, tubers were peeled and 1 cm thick cylinders were cut out. The latter were again cut into 3 mm thick slices. The fundamental experimental procedure is described in Düring et al., supra). The tuber slices arranged on a wet filter paper were pricked freshly in the center and a suspension of 2000 Erwinia carotovora ssp. atroseptica bacteria were applied in 2 ml volume. After three days, the macerated tissue was rinsed and the remaining firm potato tissue was weighed after drying it. The results of 4 transgenic lines of the MPB/aATPT series and of 3 lines of the MPB/sATPT series are shown in FIG. 1. In the antisense gene construct (lines MPB/aATPT), the content of the remaining intact tissue was about 15% for the non-transgenic control, whereas for the transgenic lines this content was approximately 90%. The sense gene construct (lines MPB/sATPT also had a content of about 35%. It is thus evident that a marked increase in the resistance, e.g. to Erwinia carotovora ssp. atroseptica can be achieved by the method according to the invention.

### EXAMPLE 2

### Increase in the Resistance of Transgenic Potato Leaves to Phytophthora infestans

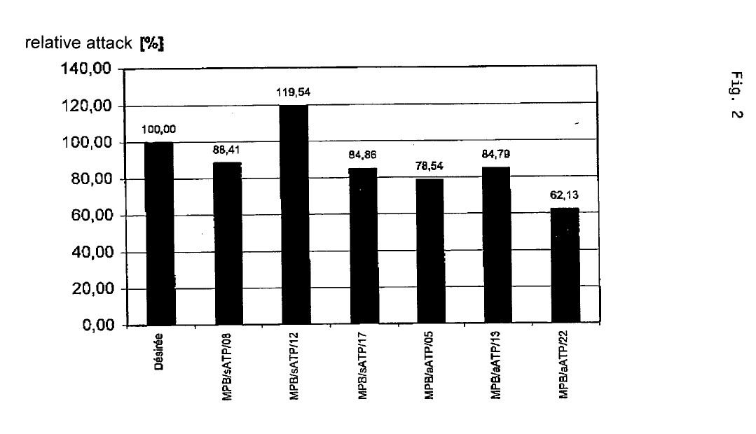

The resistance properties of the potato leaves to the pathogen Phytophthora infestans were checked by leave slice tests: Potato plants were used for this test as described in Example 1. For this purpose, round leaf slices having a diameter of 20 mm were produced from potato leaves by means of a punch. These leaf slices were arranged on a moist filter paper spread in a transparent plastic can on a stainless steel grid and inoculated with a 20 μl drop of spore suspension (about 200 sporangia) of Phytophthora infestans race 1-11. The sporangia suspension was produced by already infected leaf slices and prior to inoculation cooled to 4° C. for about 15 minutes to stimulate the zoospore hatch. The incubation was carried out in illuminated cooled incubators with a day time of 14 hours and a day/night temperature of 17/10° C. After five and six days, bonitures were made, the percentage of the attacked area as compared to the entire leaf slice area having been determined. The results of 6 transgenic lines are shown in FIG. 2.

It turned out that by using the described as constructs according to the invention it was possible to reduce the symptoms, which emphasizes the generation of pathogen resistance in plants.

### EXAMPLE 3

### Increase in the Resistance of Transgenic Potato Plants to Phytophothora infestans

For this test, the transgenic plants were also produced as described in Example 1. Phytophthora infestans was cultivated in a Petri dish (9 cm) on oatmeal/agar (Difco) at 18° C. in the dark for about 6 weeks. Then, 10 ml H₂O+0.2% gelatin (sterile) were added onto the culture, shaken and scraped off. The suspension was filtered through a filter (Miracloth) and the liquid flowing through was sprayed onto the leaves of the transgenic plant. This step was made using a spraygun (Revell) at a pressure of about 1 bar. Per plant one sprig (the last branch but one) was inoculated on the top side and bottom side of the leaf. About 1 ml of the filtered suspension was used per plant. The plants were incubated with a plastics cap in a climatic cabinet for 3 days, the temperature being 27° C. during the day (14 h) and 22° C. at night (90 to 98% relative humidity in the cabinet). Thereafter, the cap was removed. The attack was checked 48 h and 96 h after the inoculation by means of a camera.

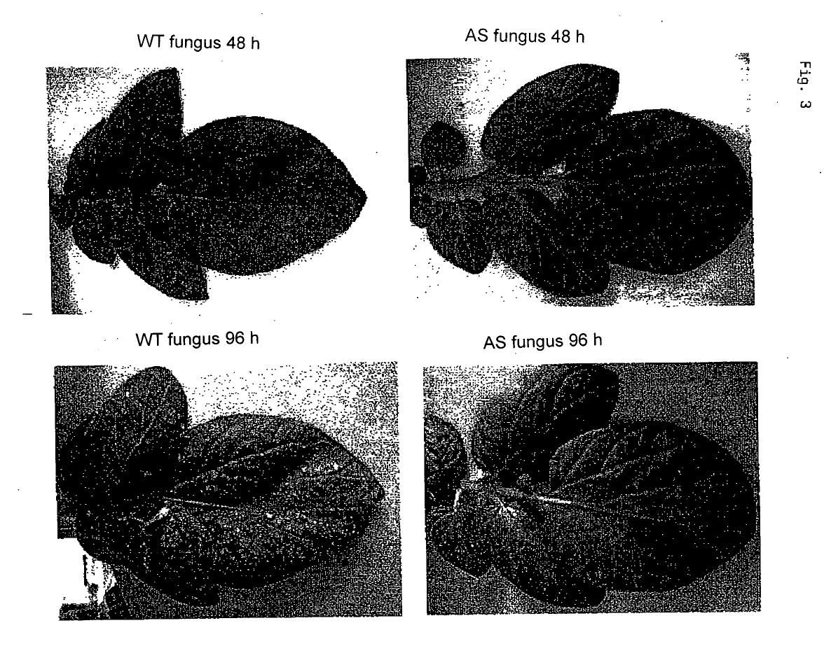

FIG. 3 shows that the damage caused by the pathogen was markedly reduced in the transgenic plants. Thus, it was possible to produce a resistance of the whole plant to the pathogen Phytophthora infestans by means of the method according to the invention.

### EXAMPLE 4

### Increase in the Resistance of Transgenic Potato Plants to an Increased Salt Concentration

The transgenic potato plants used were produced as described in Example 1. The transgenic plants were showered daily with water containing different concentrations of NaCl. The concentrations 0, 5, 10, 20 and 50 mM NaCl were used. Due to a constant supply of electrolyte in the water there was a gradual accumulation in the culture substrate of the plant. The accumulation of the electrolyte in the culture substrate was followed by measuring the conductivity. Suitable methods of determining the conductivity are known to the person skilled in the art. The resistance was evaluated by optically checking the plants. From a conductivity of 1.8 dS/m necrotic leaf regions and attack of the leaves were observed in the control plants. These symptoms occurred in the transgenic plants with markedly increased conductivity values. Up to a conductivity of 2.5 dS/m no changes in the plants were observed. Some of the above described symptoms could occur to a minor extent above this value. From a conductivity of 4.5 dS/m the transgenic plants also showed marked necroses of the leaves and attack of the leaves.

It was possible to achieve an increase in the resistance of potato plants to salt stress by the method according to the invention in this case.

1. A method of generating or increasing a resistance in an organism to biotic or abiotic stress factors, characterized in that the distribution of ATP and/or ADP in cells of the organisms is changed.

2. The method according to claim 1, wherein the organism is a plant.

3. The method according to claim 2, wherein the plant comprises gramineae, chenopodiums, leguminous plants, brassicaceae, solanaceae, fungi, mosses, and algae.

4. The method according to claim 2, wherein the plant comprises wheat, barley, rice, corn, sugar beets, sugarcane, rape, mustard, oilseed rape, flax, peas, beans, lupins, tobacco and potatoes.

5. The method according to any of claims 1 to 4, wherein the resistance is a disease resistance, pest resistance, resistance to heat, cold or dryness, U.V. rays or salt stress.

6. The method according to any of claims 1 to 5, characterized in that the activity or concentration of a protein involved in the subcellular distribution of ATP and ADP is increased or reduced in the organism.

7. The method according to any of claims 1 to 6, characterized in that the expression of a gene coding for a protein involved in the subcellular distribution of ATP and/or ADP is increased or reduced in the organism.

8. The method according to claim 7, characterized in that the expression is regulated temporally, locally and inducibly.

9. The method according to claim 7 or 8, characterized in that the expression of the plastidial ATP/ADP transporter is increased or lowered.
